SEQUENCE LISTING (6 sequences)

SEQ ID NO: 1 - DNA, 2778 bp, Raphanus sativus
agaggaattggtcaactcatcaggctcatgacctgaagattacaggttcgaatcctgtccccgcactaagtaagggtttcattctgcatcactctccccgtcgttctcgacctcgcaaggtttttgaagcggccgaagcgggaagtgacaataccgcttttcttcagcacattttggatgatttgagcgaaaacggagtacaaagttcagcctttaaggaggctatgaatcaaatagggctggtggcgcagtccccacttgaccaatttgagattgtcccattgattcctatgaatatcggaaacttctatttctcattcacaaatccatctttgttcatgctgctaactctgagttttttcctacttctgattcattttattactaaaaagggaggaggaaacttagtcccaaatgcttggcaatccttggtagagcttctttatgatttcgtgctgaacctggtaaaggaacaaataggtggtctttccggaaatgtgaaacaaatgtttttcccttgcatcttggtcacttttctttttttgttattttgtaatcttcagggtatgataccttatagcttcacagtgacaagtcattttctcattactttggctctctcattttctatttttattggcattactatagtgggatttcaaagacatgggcttcattttttcagctttttattacccgcaggagtcccactgccgttagcaccttttttagtactccttgagctaatttcttattgttttcgcgcattaagcttaggaatacgtttatttgctaatatgatggccggtcatagtttagtaaagattttaagtgggttcgcttggactatgctatgtatgaatgagattttctattttataggggctcttggtcctttatttatagttcttgcattaaccggtctggaattaggtgtagctatattacaagcttatgtttttacgatcttaatctgtatttacttgaatgatgctataaatctccattaaagttcttctttctttcatttagatttataattgaacaaaagcgagggatgagagtagtgttatttagagcagttacacagcccctctccttgcagtcgagttgctaaagcacctctcctttgctgttcgagtaaacaagaaatgctcgagttactaaacaacccctagggggcccctctctgataaggaaaaaaacgaaaaaatctcaaatttatgaaaaatcgactccaatggctgttacccctgctcggtagtttgatcggtttcgttttcagacgttttctagaatcagaaggaagcgctattctgaccactacgtgcgtttcattcttcgcgctagtgagcttctccttttgttttagtcactttcgtttgaaaggaccactgagggggattctcaatctcttcttactcttttccgctgggttcgtgggatctttcatacggattgaagtcatccacctactgggtggtcaggctttgcccctcctgttgggaccttttgtttggaaggccgtagtaggggaagcacttccttctacgggccctaacggggcggagagctcctccacgtgggaggaggatccgtttgaactggacgttctcgaggaatcattctcggactccccccggcagggagtcccagacggaagagagtgaaccctcggtcaatcgacgtagctcgaggcaccccaaacggaagagggtgaaccctcggtaaatcggcggggtcccaggaagctgggcctgcgctccagctaatccagtcccttccggggggacgaagctgggccatctgtctcctatccctacagaagggatgaaatgattgggggggatagcgtggaggcgatagaacgccgccttctggctcgatttgcttatcccttatactcggacatccaattagcccacattcaagccgaaagactcttcgaggtcaaggtagagattgtgaaggtaatggctggccttgatccaacgggggattggatggggcggggagctcgggccctcgacaatccccgtaccgccacgggagagcactccgtgaagcagttataccacctgttaagtgctctaaatgagcgcggtaaagaggccccagaatttcaggaactcaaaaatagagtgttcctcaagaaaggcggccctgggggggactctatcgcataagtaagcaagctacctttcgggatgggcttttgcttctttctttatttttcgatatgccgcttcttcgccagcaaggagcgagaaaacaaagtgggctgtaatgatgtcagaatttgcaccaatttctatctatttagtgattagtctgctagtttctttgatcctactcggtgttccttttccatttgcttccaatagttctacctacccagaaaaattgtcggcctacgaatgtggtttcgatccttccggtgatgccagaagtcgtttcgatatacgattttatcttgtttcaattttatttttaatccctgatctggaagtcacctttttctttccttgggcagtacctcccaacaagattgatctgtttggattttggtccatgatggcctttttatttattttgacgattggatttctctatgagtggaaaaggggtgcttcggatcgggagtaaagtgatagggcaaaaaatggggggggggaaaaagaaaggaaagagaataatgcctacgttgaattcaattgattcgtcatggtagagaagaaaaacggcgcacggaccgtactcgagctttggataaatgtc

SEQ ID NO: 2 - DNA, 940 bp, Raphanus sativus
ctcgagtgaatgagtggtatatgcagaatttgggggttcctggccctgctaattttcttcaagaatatgaagaaaaatgttactctaactatgttagagtgatggaaatccccacacccctggaagaatggaatttgacatttttatttactattactactatcggtattatattattatttttcctatttttgggtatgagaagaaagaagaattgcaagaagtgtgagaaagggatgttagaaggtgcaaaatcaataggtgccggagctgctacaattgcttcagcgggagctgctgtcggtattggaaacgtcttcagttctttgatccattctgtggcgcgaaatccatcattggctaaacaatcatttggttatgccattttgggctttgctctaaccgaagctattgcattgtttgccccaatgatggcctttttgatcttattcgtattctgatcgaagaaagaaggtttcattcagtctcataaagcaagcacctctttcacataagaaagtggaggcaggcttggatacgatctaaaatgattccacatgaaagaggaccgggcaatcgccctcttgagtaatgggaagcgggctagttcccgaaaatgcccgttaatcaagcaagttggggacaaaatttgccttataatcaaataaggcttcagcgttgtaacaagccttctgagccaggatcaaactcctccttttgaaagagttccaccgcccgcctactcccccccccccgcgccagcctctgttccagcctgtcagctagtgaagtccgtttcctaacctaagggagtaggctttgcctaggaggacaactattatatgttttcaactgagttctactgttcacctacaacccaacctaatcttactgaaactcttgcttttccttgttatctttcttttgcctatcgaagctt

SEQ ID NO: 3 - DNA, 23 bp, Artificial Sequence
  primer
cgcttggactatgctatgtatga

SEQ ID NO: 4 - DNA, 29 bp, Artificial Sequence
  primer
ttttccactcatagagaaatccaatcgtc

SEQ ID NO: 5 - DNA, 33 bp, Artificial Sequence
  primer
ctcgagtgaatgagtggtatatgcagaatttgg

SEQ ID NO: 6 - DNA, 21 bp, Artificial Sequence
  primer
ctgacaggctggaacagaggc

(19) United States
(12) Patent Application Publication
(10) Pub. No.: US 2017/0367289 A1
(43) Pub. Date: Dec. 28, 2017

(54) NWB-CMS BRASSICA OLERACEA HAVING CYTOPLASMIC MALE STERILITY AND USE THEREOF

(71) Applicant: NONGWOO BIO CO., LTD, Suwon-si, Gyeonggi-do (KR)

(72) Inventors: Chee Hark HARN, Gyeonggi-do (KR); Min JUNG, Gyeonggi-do (KR); In Chang JANG, Gyeonggi-do (KR); Jong Sep SHIN, Gyeonggi-do (KR); Shi Woo LEE, Gyeonggi-do (KR); Dong-Su IN, Gyeonggi-do (KR); Gyun Young JOO, Gyeonggi-do (KR)

(21) Appl. No.: 14/386,701

(22) PCT Filed: Jan. 21, 2013
(86) PCT No.: PCT/KR2013/000448

§ 371 Date: Sep. 19, 2014

(30) Foreign Application Priority Data
Mar. 21, 2012 (KR) 10-2012-0028632

(51) Int. Cl. A01H 5/10; C12N 15/02

(52) U.S. Cl. CPC A01H 5/10; C12N 15/02

(57) ABSTRACT

NWB-CMS Brassica oleracea has cytoplasmic male sterility. It relates to NWB-CMS cabbage plant having cytoplasmic male sterility derived from NWB-CMS cabbage line produced by fusion between protoplast of NWB-CMS radish line plant derived from callus of NWB-CMS radish line having cytoplasmic male sterility with inactivated nucleus and protoplast of male fertile cabbage plant with inactivated cytoplasm and a seed thereof, a plant of NWB-CMS Brassica oleracea line having cytoplasmic male sterility produced by breeding of a male fertile Brassica oleracea as a subject for introduction with the NWB-CMS cabbage plant as a breeding line and a seed thereof, a method for producing a hybrid seed of NWB-CMS Brassica oleracea line having cytoplasmic male sterility comprising breeding of a male fertile Brassica oleracea as a subject for introduction with the NWB-CMS cabbage plant as a breeding line, and a hybrid seed of NWB-CMS Brassica oleracea line produced by the method.

### CROSS REFERENCE TO RELATED APPLICATIONS AND CLAIM OF PRIORITY

This patent application is a National Phase application under 35 U.S.C. §371 of International Application No. PCT/KR2013/000448, filed Jan. 21, 2013, which claims priority to Korean Patent Application No. 10-2012-0028632, filed Mar. 21, 2012, entire contents of which are incorporated herein by reference.

### BACKGROUND

### Technical Field

The present invention relates to NWB-CMS Brassica oleracea having cytoplasmic male sterility and use thereof. More specifically, it relates to NWB-CMS cabbage plant having cytoplasmic male sterility which is derived from NWB-CMS cabbage line produced by fusion between protoplast of NWB-CMS radish line plant derived from callus of NWB-CMS radish line having cytoplasmic male sterility with inactivated nucleus and protoplast of male fertile cabbage plant with inactivated cytoplasm and a seed thereof, a plant of NWB-CMS Brassica oleracea line having cytoplasmic male sterility which is produced by breeding of a male fertile Brassica oleracea as a subject for introduction with the NWB-CMS cabbage plant as a breeding line and a seed thereof, a method for producing a hybrid seed of NWB-CMS Brassica oleracea line having cytoplasmic male sterility comprising breeding of a male fertile Brassica oleracea as a subject for introduction with the NWB-CMS cabbage plant as a breeding line, and a hybrid seed of NWB-CMS Brassica oleracea line produced by the aforementioned method.

### Background Art

CMS represents cytoplasmic male sterility and it is a phenomenon of lacking self fertilizing activity as caused by sterility based on a problem in male organ such as pollen, anther, stamen or the like. When mitochondria cannot perform a regular function due to cytoplasmic problems, it may result in an abnormal fertile activity. CMS belongs to maternal inheritance. It is very easy to maintain a male sterile system and application to crops which use vegetative organs like leaf, stem, or the like is very convenient. Collecting a seed and establishing a production system using the male sterility are the most important object of using CMS, and it is a dream of many breeders to produce many lines having such traits. For such reasons, many attempts have been made to use CMS for producing F₁ seed, and studies have been made on CMS of various plants.

In particular, world-renowned multinational seed companies have tried to develop a breeding system for cruciferous crops by using CMS technology. However, no male sterility has been identified for cruciferous crops until 1990 except leaf mustard and radish. As such, there are many restrictions for using it for collecting seeds. Since then, many CMS systems have been found and used, and production of a hybrid seed by using Ogura CMS line is most widely performed. However, when Ogura CMS is used as a breeding line, it is known that CMS state is unstable in some breeding lines, and thus it was indicated that the production of F₁ hybrid seed using Ogura CMS line has its own limit.

Nongwoo Bio Co., Ltd. has succeeded in developing and lineaging native NWB-CMS from radish. Further, the invention of a molecular marker for identifying NWB-CMS was granted with patent right, together with the NWB-CMS line (Korean Patent Registration No. 0399333). It is a unique system which is distinguished from CMSs that are conventionally and mainly used in foreign countries. Specifically, compared to Ogura CMS line, it exhibits higher stability among different generations and has less phenomenon of unstable CMS trait caused by an environment factor, and thus it has a huge advantage for producing F₁ seed. When Ogura CMS radish line is experimentally used as a breeding line, the male sterile line was yielded only from about 50% of sixteen kinds of breeding lines. It was also possible to confirm that there is a significant difference in the presence or absence of CMS introduction of Ogura CMS radish line among different lines. On the other hand, when the NWB-CMS radish line developed by Nongwoo Bio Co., Ltd. is used as a breeding line, it was possible to confirm that all sixteen kinds of breeding line yield male sterile line and also 100% male sterile line is yielded from the breeding test. As such, it was possible to confirm that the male sterility introduction ratio by NWB-CMS is significantly better than conventional Ogura CMS radish line (Nahm et al., (2005) Theo Appl Genet 111: 1191-1200). As such, radish development is currently all based on NWB-CMS and very promising breeds have been developed during last several years.

In Korean Patent Application Publication No. 1997-0073319, “Cytoplasmic male sterile Brassica oleracea plant and method for producing the same” is disclosed. In Korean Patent Application Publication No. 1999-0044535, “Cytoplasmic male sterile Brassica oleracea plant which contains polima cytoplasmic male sterile cytoplasm and is male sterile at high temperature and low temperature” is described. However, no descriptions have been made therein regarding the NWB-CMS Brassica oleracea having cytoplasmic male sterility and use thereof as described in the present invention.

### SUMMARY

The present invention is devised in view of the needs described above, and inventors of the present invention confirmed that NWB-CMS cabbage plant derived from NWB-CMS cabbage line, which is produced by fusion between protoplast of NWB-CMS radish plant having cytoplasmic male sterility with inactivated nucleus and protoplast of male fertile cabbage plant with inactivated cytoplasm, has cytoplasmic male sterility (CMS) and also confirmed that a NWB-CMS Brassica oleracea having cytoplasmic male sterility or a hybrid seed of NWB-CMS Brassica oleracea can be produced by breeding of a male fertile Brassica oleracea as a subject for introduction (e.g., broccoli, cauliflower, kohlrabi, or the like) with the aforementioned NWB-CMS cabbage plant as a breeding line. The present invention is completed accordingly.

To solve the aforementioned problems, the present invention provides NWB-CMS cabbage plant having cytoplasmic male sterility which is derived from NWB-CMS cabbage line produced by fusion between protoplast of NWB-CMS radish line having cytoplasmic male sterility with inactivated nucleus and protoplast of male fertile cabbage plant with inactivated cytoplasm, and a seed thereof.

The present invention also provides a plant of NWB-CMS Brassica oleracea line having cytoplasmic male sterility which is produced by breeding of a male fertile Brassica oleracea as a subject for introduction with the NWB-CMS cabbage plant as a breeding line, and a seed thereof.

The present invention also provides a method for producing a hybrid seed of NWB-CMS Brassica oleracea line having cytoplasmic male sterility comprising breeding of a male fertile Brassica oleracea as a subject for introduction with the NWB-CMS cabbage plant as a breeding line, and a hybrid seed of NWB-CMS Brassica oleracea line produced by the aforementioned method.

Cytoplasmic male sterility (CMS) belongs to F₁ breeding system and it is a technology which is important from the viewpoint of obtaining purity of a seed and production of a seed. It may provide a base for obtaining huge economic profits in the F₁ seed market of cruciferous plants, in particular. In other words, it may trigger the establishment of a breeding system that is important to increase national competitiveness. In year 2011, Korean vegetable seed market is estimated to be about 230 billion Korean Won. The seed market for cruciferous crops corresponds to about 50 billion Korean Won, indicating that it is quite important in Korea. Further, the international seed market for cruciferous crops corresponds to about 0.5 billion US Dollars, representing a very large market. Because most of the breeding system or technology relating to seed development or production of cruciferous crops is currently based on CMS, it can be said that seed sales basically depend on a good breeding technique using CMS. There have been many good varieties developed for cabbage, broccoli, and cauliflower, which are a main western crop of the present invention. As such, the sales competition is extremely high among varieties each owned by many multinational companies. As such, once genetic resources with new CMS trait are developed, a significant change in the market is expected.

Based on production of good F₁ seed at low cost and high purity by using Korea's own NWB-CMS, it is expected that NWB-CMS cabbage, NWB-CMS broccoli, NWB-CMS cauliflower and NWB-CMS kohlrabi developed by the present invention may have a superior position in competition with foreign companies. Further, because it is enabled by the present invention to efficiently control the worldwide problem of copying good F₁ seed by using previously established molecular marker in an international market and the outflow of genetic resources and the intellectual property right, there is an advantage of having global monopoly over cruciferous crops with NWB-CMS trait.

### BRIEF DESCRIPTION OF THE DRAWINGS

FIG. 1 is a diagram illustrating the development of cabbage cytoplasm fusion plant (cybrid) having CMS trait of radish.

FIG. 2 illustrates isolated protoplast of radish and cabbage.

FIG. 3 illustrates culture and regeneration of protoplast of cabbage (A, protoplast isolated from cabbage seed leaf; B and C, proceeding of protoplast isolation; D, callus produced from protoplast (after culture for 38 days); shoot regenerated from callus; and F, cabbage grown from shoot).

FIG. 4 illustrates over-the-time progress of isolated protoplast after treatment with IOA or γ-ray (A, cabbage protoplast treated with IOA (Iodoacetate) (Day 1); B, radish protoplast treated with γ-ray (Day 1); C, cabbage protoplast 7 days after the treatment with IOA; D, radish protoplast 7 days after the treatment with γ-ray). With no fusion, all lost the activity and were perished.

FIG. 5 illustrates an apparatus for electric fusion (A, Electro cell manipulator (ECM 2001; BTX, Inc, San Diego, USA); B, Ts100 microscope (Nikon, Tokyo, Japan); C, a fusion chamber for carrying out electric fusion under microscope. The electric fusion is performed for 3 to 8 seconds at 260-280V; D, 1.5 ml protoplast mix (2×10⁶ mix/ml) can be applied to each lane of the fusion chamber, and there are five lanes in total).

FIG. 6 illustrates the progress of electric fusion of protoplast (A, protoplasts arranged like a pearl necklace in an electric field; B, cells under fusion; C, fused cells; D, various fusion products with presumed fusion).

FIG. 7 illustrates the process in which a callus is formed from fused cells and shoots are generated from the callus (A, occurrence of callus after fusion; B, shoot formation is induced by selecting only the fused callus after determination of a fusion product among calluses by using a marker; C, shoot formation).

FIG. 8 illustrates a process of examining the callus from radish-cabbage fusion by PCR using NWB-CMS marker. Half of the callus produced from the fusion product was analyzed by PCR, and after selecting only the callus with CMS marker, shoot formation was induced.

FIG. 9 illustrates the process of examining the presence or absence of CMS trait by using two markers followed by selection for determining again the fusion product from cabbage shoots, and cultivating it again (A, many shoots were examined by using NWB-CMS1 marker and NWB-CMS3 marker, 1-24: shoots with fusion, 25-26: Ogura CMS cabbage, 27-28: cabbage control group, 29-30: NWB-CMS radish (positive control group), 31-32: MF radish control group; B, photographic images for comparing the cabbage with no fusion to the cabbage with fusion (cabbages are two months after transplant in a pot)).

FIG. 10 illustrates a flower structure of the cabbage with fusion. Both the fusion products 3A and EF3 were confirmed to be MS (male sterile).

FIG. 11 illustrates the progress of bolting after breeding between the cabbage with fusion and a Brassica oleracea (A, cabbage; B, kohlrabi; C, cauliflower; D, broccoli).

FIG. 12 illustrates a flower structure after breeding between the cabbage with fusion and a Brassica oleracea. All were confirmed to be MS (male sterile).

### DETAILED DESCRIPTION

In order to achieve the purpose of the present invention, the present invention provides NWB-CMS cabbage plant having cytoplasmic male sterility derived from NWB-CMS cabbage line produced by fusion between protoplast of a plant of NWB-CMS radish line, which is derived from a callus (deposit number: KCTC 10339BP) of NWB-CMS radish line having cytoplasmic male sterility with inactivated nucleus, and protoplast of male fertile cabbage plant with inactivated cytoplasm, and a seed thereof.

On Sep. 18, 2002, the callus of NWB-CMS radish line was duly deposited with Korean Collection for Type Cultures (KCTC) (having the address of Biological Resource Center (BRC), Korea Research Institute of Bioscience and Biotechnology (KRIBB), 52 Eoeun-dong, Yuseong-gu, Daejeon 305-333, Republic of Korea) under the Access number of KCTC 10339BP. The deposit has been made under the terms of the Budapest Treaty and all restrictions imposed by the depositor on the availability to the public of the biological material will be irrevocably removed upon the granting of a patent.

The callus of NWB-CMS radish line having cytoplasmic male sterility and the plant of NWB-CMS radish line derived from the callus of the present invention are described in Korean Patent Registration No. 0399333.

The male sterility due to a cytoplasmic factor as described in the present invention is referred to as cytoplasmic male sterility (herein after, ‘CMS’) and it indicates a phenomenon in which mitochondria cannot perform a normal function due to a cytoplasmic problem and produces abnormal pollen, yielding no self-fertilization activity. CMS is maternal inheritance, and 100% sterile line is always yielded regardless of using any specific fertilization system. Thus, there is an advantage that the male sterile system can be easily maintained and it can be applied conveniently to crops which use vegetative organs such as leaf or stem.

The NWB-CMS cabbage plant according to the present invention can be subjected to an asexual reproduction based on a common tissue culture method that is well known in the pertinent art. For example, asexual reproduction can be achieved by micropropagation using organ generation, which is useful for tissue culture of a cruciferous plant like Chinese cabbage and cabbage (i.e., a method of inducing fresh buds on a tissue surface by culturing tissues like leaf, leaf stalk, stem node, seed leaf, seed leaf stem, or the like) or a regeneration method based on callus induction. Specifically, in the present invention, the seed was added and cultured on ½ MS medium, and seed leaves having some leafstalks are removed and then regenerated on MS medium for callus induction. After that, shoot formation from the callus was induced and a callus with formed shoot was transferred to a medium for inducing root formation. After undergoing soil acclimation and regeneration processes, it is grown to a mature plant.

The NWB-CMS cabbage plant according to the present invention is a cytoplasm fusion plant produced by fusion between protoplast of a plant of NWB-CMS radish line, which is derived from a callus of NWB-CMS radish line having cytoplasmic male sterility with inactivated nucleus, and protoplast of a male fertile cabbage plant with inactivated cytoplasm.

In the present invention, the “plant” includes a plant organ, a plant tissue, a plant cell, a seed, and a callus.

With regard to the plant according to one embodiment of the present invention, the NWB-CMS cabbage plant may contain NWB-CMS1 marker or NWB-CMS3 marker, which consists of a nucleotide sequence of SEQ ID NO: 1 and SEQ ID NO: 2, respectively.

With regard to the plant according to one embodiment of the present invention, each of NWB-CMS1 marker and NWB-CMS3 marker may be preferably amplified with a primer set consisting of a nucleotide sequence of SEQ ID NO: 3 and SEQ ID NO: 4 or a primer set consisting of a nucleotide sequence of SEQ ID NO: 5 and SEQ ID NO: 6, but the invention is not limited to them.

NWB-CMS1 marker consisting of a nucleotide sequence of SEQ ID NO: 1 of the present invention is described as the NWB-CMS marker in Korean Patent Registration No. 0399333.

NWB-CMS3 marker consisting of a nucleotide sequence of SEQ ID NO: 2 of the present invention is newly isolated as a DNA marker for selecting a plant with cytoplasmic male sterility from the mitochondria genome of a plant of NWB-CMS1 radish plant.

The present invention also provides a plant of NWB-CMS Brassica oleracea line having cytoplasmic male sterility which is produced by breeding of a male fertile Brassica oleracea as a subject for introduction with the NWB-CMS cabbage plant as a breeding line, and a seed thereof.

With regard to the plant of NWB-CMS Brassica oleracea line according to one embodiment of the present invention, the Brassica oleracea can be any one of broccoli, cauliflower, and kohlrabi, but it is not limited to them.

The male fertile Brassica oleracea as described in the present invention indicates broccoli, cauliflower, or kohlrabi which allows normal natural breeding.

With regard to the plant according to one embodiment of the present invention, the NWB-CMS cabbage plant may contain NWB-CMS1 marker or NWB-CMS3 marker, which consists of a nucleotide sequence of SEQ ID NO: 1 and SEQ ID NO: 2, respectively.

With regard to the plant according to one embodiment of the present invention, NWB-CMS1 marker or NWB-CMS3 marker may be amplified with a primer set consisting of a nucleotide sequence of SEQ ID NO: 3 and SEQ ID NO: 4 or a primer set consisting of a nucleotide sequence of SEQ ID NO: 5 and SEQ ID NO: 6, but the invention is not limited to them.

The present invention also provides a method for producing a hybrid seed of NWB-CMS Brassica oleracea line having cytoplasmic male sterility comprising breeding of a male fertile Brassica oleracea as a subject for introduction with the NWB-CMS cabbage plant as a breeding line, and a hybrid seed of NWB-CMS Brassica oleracea line produced by the aforementioned method.

Specifically, according to breeding between a plant of NWB-CMS Brassica oleracea line of the present invention and a plant desired for introduction of male sterility (i.e., male fertile Brassica oleracea line), F₁ hybrid seed can be produced. The method of the present invention can be applied to any male fertile broccoli, cauliflower, or kohlrabi, but the present invention is not limited to them.

Herein below, the present invention is explained in view of the examples. However, the following examples are given only to illustrate the present invention and by no means the scope of the present invention is limited to them.

### Example 1: Isolation of Protoplast

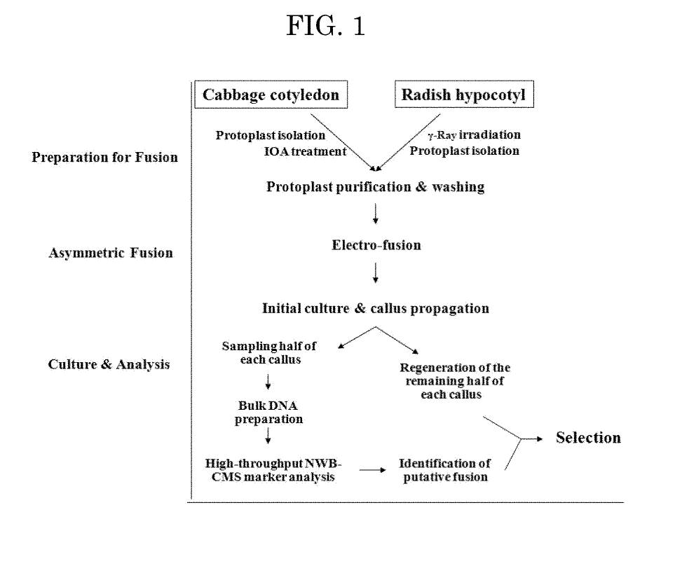

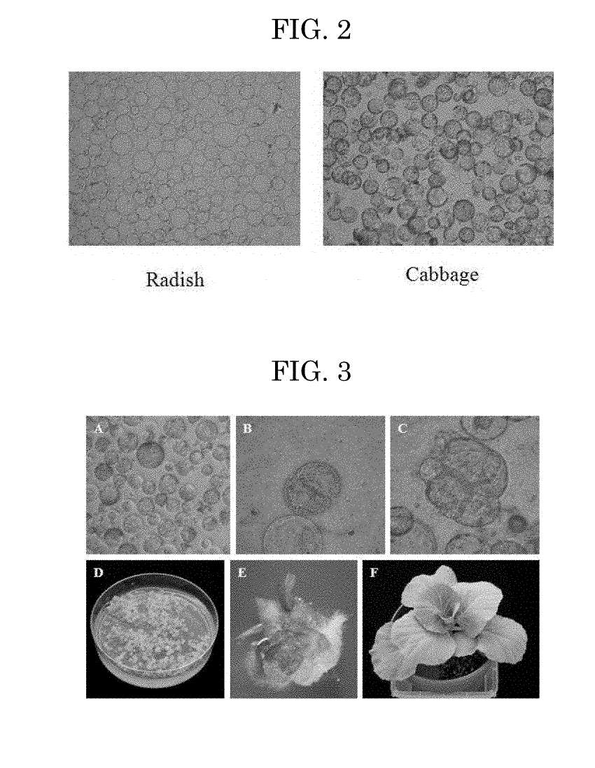

An asymmetric cell fusion method for transferring NWB-CMS trait of radish to cabbage and a road map for culture were constructed (FIG. 1). As a plant material, a specimen of cabbage seed leaf (i.e., cabbage cotyledon) or radish hypocotyl that are 7 to 10 days after germination were used. The radish is derived from a callus (deposit number: KCTC 10339BP) of NWB-CMS radish line described in Korean Patent Registration No. 0399333. Protoplast was isolated from seed leaf of the cabbage and treated with IOA (Iodoacetate). Radish hypocotyl was irradiated with γ-ray, and then the protoplast was isolated from it. For isolation of a large amount of protoplast, the optimum enzyme composition was established. Specifically, isolation conditions were optimized by having the ratio of 1:0.5:0.5 among viscozyme:cellcalst:pectinex. By using those conditions, it was confirmed that the separation degree of the protoplast in seed leaf or hypocotyl of the plant is between 1×10⁷ and 1×10⁸ or so per gram (FIG. 2).

### Example 2: Culture and Regeneration of Cabbage Protoplast

Establishing a regeneration system for producing a plant from cabbage protoplast was a prerequisite for obtaining a fusion product between cabbage nucleus and radish cytoplasm. Cabbage seeds were washed by impregnating them in 70% ethanol followed by surface sterilization for 10 minutes in 50% Clorox solution and washing with sterilized water. After that, they were sown on MSB5 medium as a seed sowing medium. After germination for 5 days under dark conditions, they were transferred to light conditions and cultured for 2 days. Seed leaf with recovered chlorophyll amount was used. For protoplast isolation, leaf tissues were cut, added to a petri dish, and treated for 12 hours at 27° C. and 40 rpm in a medium comprising three kinds of enzyme (4% viscozyme, 2% cellcalst, and 2% pectinex). The isolated protoplast and cells were filtered using a sieve with pore size of 80 μm and centrifuged in a 15 ml tube for 5 minutes at 800 rpm. The isolated protoplast was washed two or three times using W5 (310 mM NaCl, 20 mM CaCl₂.2H₂O, 10 mM glucose, 10 mM KCl, 3 mM MES, pH 5.6) followed by culture at concentration of 5 to 10×10⁵/ml in induction medium BDG (MS salt, 2% glucose, 7% mannitol and 0.5 to 1 mg/L BAP, 1 to 3 mg/L 2,4-D). The cabbage protoplast isolated from seed leaf (FIG. 3A) starts to show cell division from Day 4 after initial culture step and is developed into a multi-cellular body on Day 15 (FIGS. 3B and C). The multi-cellular body after liquid culture and microcalluses were transferred to and cultured in a proliferation medium (MS, 2 to 5 mg/L chinetin, 0.5 mg/L 2,4-D, 3% sucrose, agar 0.8%, pH 5.8) (FIG. 3D). The proliferated callus mass was separated and cultured in a medium for inducing shoot (MSB5 0.5 to 2 mg/L BAP, 3% sucrose, agar 0.8%, pH 5.8) (FIG. 3E), and then a cabbage plant was produced after an acclimation process (FIG. 3F).

### Example 3: Fusion of Isolated Protoplast

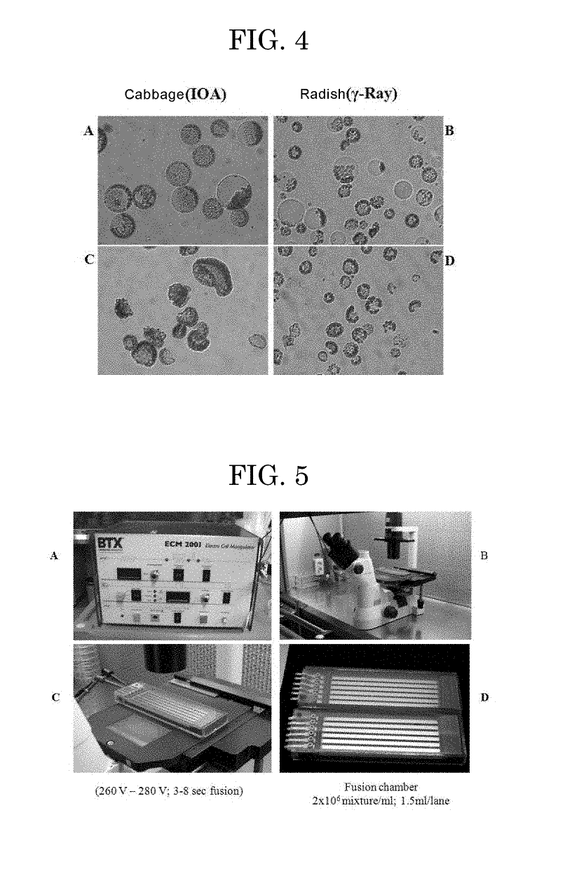

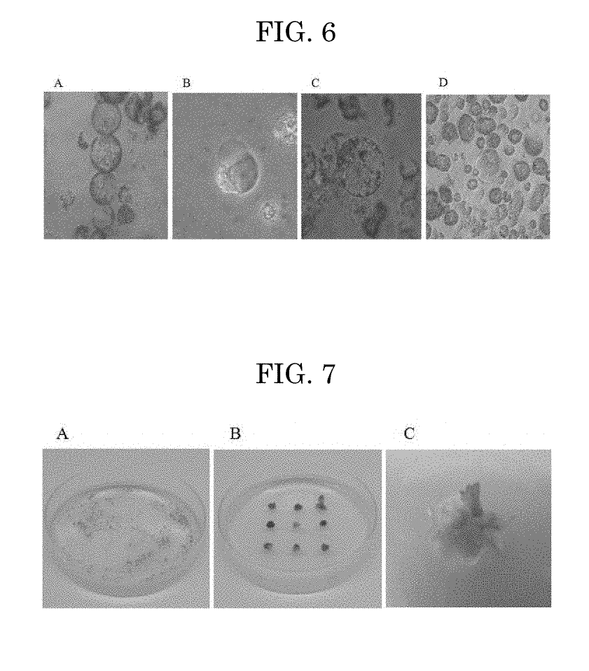

Protoplast isolated from the cabbage seed leaf was treated with 3 to 10 mM IOA (Iodoacetate) for 10 minutes to suppress differentiation of mitochondria in cytoplasm. The protoplast isolated from radish hypocotyl was treated with gamma ray having irradiation amount of 0.5 to 5 kGy by using ⁶⁰CO (1.33 MeV) to inactivate the DNA of radish nucleus. Seven days after the treatment with IOA of normal cabbage protoplast (FIG. 4A), the protoplast was perished in distorted shape without showing any cell division (FIG. 4C). Further, the protoplast separated from radish hypocotyl (FIG. 4B) was also perished without showing any cell division, seven days after the treatment with gamma ray (FIG. 4D). Protoplasts of radish and cabbage which have been treated as above were subjected to inactivation of nucleus and cytoplasmic mitochondria for performing asymmetric cell fusion. For electric fusion, protoplasts of radish and cabbage were admixed with each other at ratio of 1 to 3:1. After that, they were transferred to a fusion chamber at concentration of 2 to 5×10⁵/ml using buffer for electric cell fusion (E10: 9% mannitol, 0.5 mM CaCl₂, 3 mM MES, pH 5.8) for stabilization. As an apparatus for electric cell fusion, ECM 2001 (BTX, Inc, San Diego, USA), Ts100 microscope (Nikon, Tokyo, Japan), and a self-assembled fusion chamber (1.5 ml/lane, 5 lanes/chamber) were used (FIG. 5A to D). The protoplast in fusion chamber was subjected to cell sorting using AC current (15 to 80 V/cm, 3 to 50 seconds) (FIG. 6A), and then the cell fusion of the two cells was performed with DC current (pulse: 180 to 320 V/cm, 60 μsec) (FIG. 6B). After the fusion, the inducing medium BDG was added and cells were cultured in a liquid state at concentration of 2 to 5×10⁵/ml. The fused cells exhibited enlarged shape 6 days after the first culture (FIGS. 6C and D).

### Example 4: Shoot Generation from Fusion Product

The protoplasts each isolated from cabbage seed leaf and radish hypocotyls were subjected to electric cell fusion, and 4 weeks after the initial culture, a multicellular body and microcalluses were formed via cell division. They were subcultured using a proliferation medium (MS, 2 to 5 mg/L chinetin, 0.5 mg/L 2,4-D, 3% sucrose, agar 0.8%, pH 5.8) (FIG. 7A). After that, the small fused calluses were proliferated (FIG. 7B) and added to a medium for inducing shoot (MSB5, 0.5 to 2 mg/L BAP, 3% sucrose, agar 0.8%, pH 5.8) for producing a small plant (FIG. 7C).

### Example 5: Selection of Fused Cabbage Product by Using NWB-CMS Marker

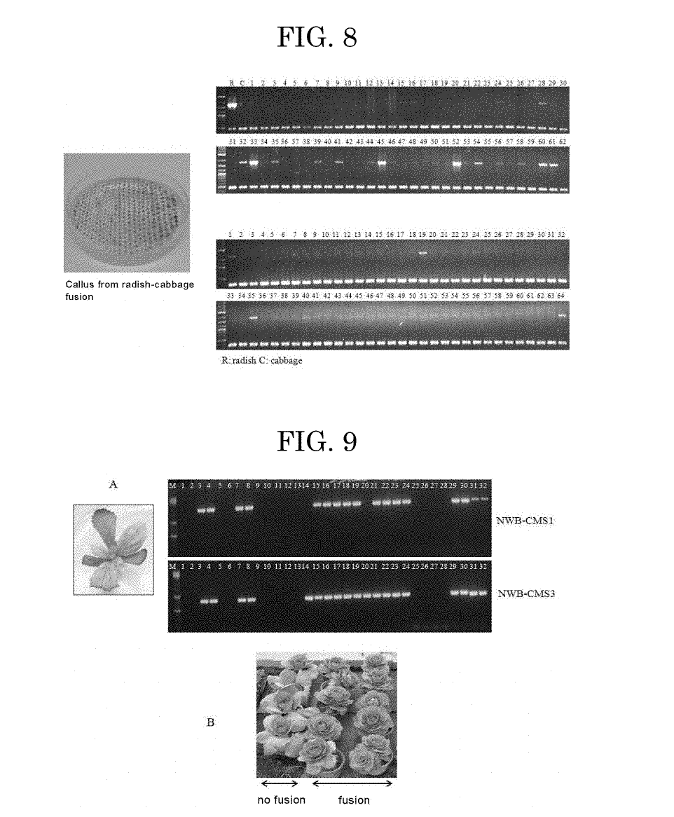

In order to confirm the fusion, the calluses derived from the fusion product were divided into two parts, and DNA was extracted from one of them and determined by PCR using NWB-CMS marker (FIG. 8). Only the calluses which have been selected by using the marker were added to a medium for inducing shoot (MSB5, 0.5 to 2 mg/L BAP, 3% sucrose, agar 0.8%, and pH 5.8) for shoot growth. NWB-CMS1 marker was identified as 1.8 kb band as a result of performing PCR using forward primer 5′-cgcttggactatgctatgtatga-3′ (SEQ ID NO: 3) and a reverse primer 5′-ttttccactcatagagaaatccaatcgtc-3′ (SEQ ID NO: 4). PCR conditions are as follows: 94° C., 30 seconds; 60° C., 30 seconds; 72° C., 90 seconds; 35 cycles.

Actual incorporation of NWB-CMS in the induced cabbage shoot was determined again by using two markers, i.e., NWB-CMS1 and NWB-CMS3 (FIG. 9A). PCR conditions for identifying the NWB-CMS1 marker are as described above, and NWB-CMS3 marker was identified as 0.78 kb band as a result of performing PCR using forward primer 5′-ctcgagtgaatgagtggtatatgcagaatttgg-3′ (SEQ ID NO: 5) and a reverse primer 5′-ctgacaggctggaacagaggc-3′ (SEQ ID NO: 6). PCR conditions are as follows: 94° C., 30 seconds; 60° C., 30 seconds; 72° C., 90 seconds; 35 cycles. Only the shoots having the two markers at the same time were collected and transferred to a pot, and eventually grown in a green house (FIG. 9B).

### Example 6: Efficiency for Obtaining Fused Cabbage Product

Number of the selected calluses which is determined to be PCR positive is 1.3% of initial protoplast number of 3×10⁵. Since the regeneration efficiency of cabbage is about 5% (data not shown) and the efficiency of having shoots that are proven to be PCR positive is about 54%, the probability for obtaining the final fused shoot is about 0.04%. Thus, when protoplasts are used in a number which is 100 times larger than 3×10⁵, the fused shoots can be obtained at a rate of about 4%. Further, since the number 3×10⁷ corresponds to a protoplast mixture of about 30 ml in a fusion chamber, it can be said that relatively high efficiency has been obtained.

TABLE 1
Efficiency for obtaining fused cabbage product

| | | Ratio of | Callus number | Selected | |
|---|---|---|---|---|---|
| Method for | Number of | producing | determined | callus | |
| fusion | protoplast | callus | by marker | PCR+ | % |
| Electric | 3 × 10⁵ | 0.082 ± 0.003 | 1004 | 13 | 1.3 |
| fusion | | | | | |

### Example 7: Flower Structure of Fused Cabbage Product and Breeding

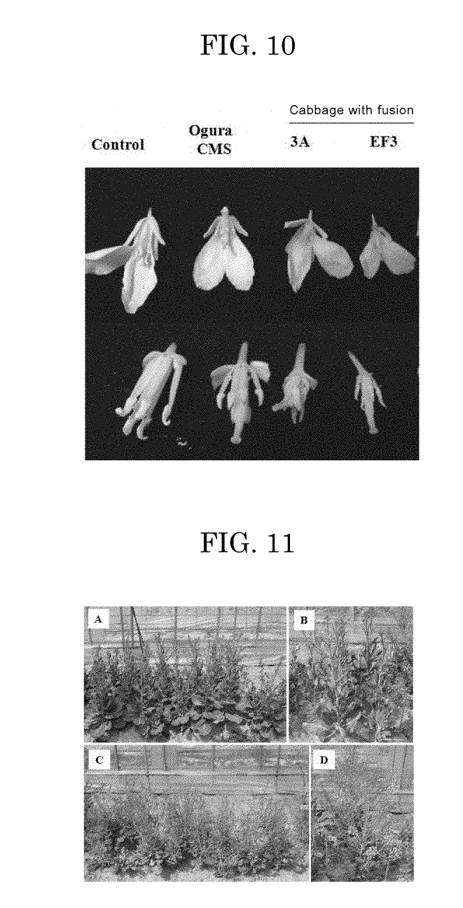

The fusion product was cultivated and the flower structure was observed after bolting. Both the fused cabbage 3A and EF3 did not form any pollen (FIG. 10) and they are similar to the shape of Ogura CMS which is already used for a breeding program. On the other hand, the non-fusion product yielded well developed pollen. Horticuturally, the fused cabbage overall has the same morphology as normal cabbage, but there are also several plants which exhibit various morphological changes.

In order to develop CMS Brassica oleracea, the horticulturally most favorable CMS fusion product of cabbage was chosen and subjected to breeding with other normal cabbage, broccoli, cauliflower, or kohlrabi. The bolting process has also successfully occurred during the following cultivation (FIG. 11A: cabbage; B: kohlrabi; C: cauliflower; D: broccoli). The breeding was performed by a typical method for breeding plants.

### Example 8: Flower Structure of Brassica oleracea

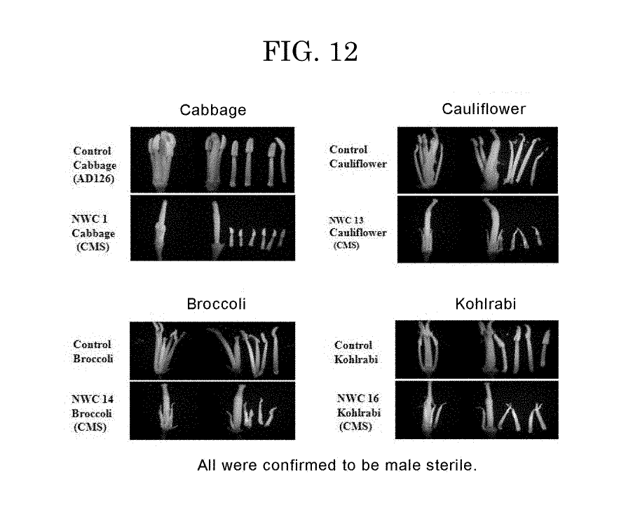

Flower structure of the Brassica oleracea which have been bred with CMS fusion cabbage was examined. As a result, it was found that all exhibit no pollen formation (100%) (FIG. 12). This means that the CMS trait is transferred from the fusion cabbage to other Brassica oleracea, and thus it was able to develop new genetic resources of a Brassica oleracea having CMS trait.

1. NWB-CMS cabbage plant having cytoplasmic male sterility which is derived from NWB-CMS cabbage line produced by fusion between protoplast of NWB-CMS radish line plant derived from callus (deposit number:KCTC 10339BP) of NWB-CMS radish line having cytoplasmic male sterility with inactivated nucleus and protoplast of male fertile cabbage plant with inactivated cytoplasm.

2. The NWB-CMS cabbage plant according to claim 1, characterized in that the NWB-CMS cabbage plant comprises NWB-CMS1 marker or NWB-CMS3 marker which consists of a nucleotide sequence of SEQ ID NO: 1 and SEQ ID NO: 2, respectively.

3. The NWB-CMS cabbage plant according to claim 2, characterized in that the NWB-CMS1 marker or NWB-CMS3 marker is amplified with a primer set consisting of a nucleotide sequence of SEQ ID NO: 3 and SEQ ID NO: 4 or a primer set consisting of a nucleotide sequence of SEQ ID NO: 5 and SEQ ID NO: 6, respectively.

4. A seed of the NWB-CMS cabbage plant according to claim 1.

5. A plant of NWB-CMS Brassica oleracea line having cytoplasmic male sterility which is produced by breeding of a male fertile Brassica oleracea as a subject for introduction with the NWB-CMS cabbage plant of claim 1 as a breeding line.

6. The plant of NWB-CMS Brassica oleracea line according to claim 5, characterized in that the Brassica oleracea is broccoli, cauliflower, or kohlrabi.

7. The plant of NWB-CMS Brassica oleracea line according to claim 5, characterized in that the NWB-CMS cabbage plant comprises NWB-CMS1 marker or NWB-CMS3 marker which consists of a nucleotide sequence of SEQ ID NO: 1 and SEQ ID NO: 2, respectively.

8. The plant of NWB-CMS Brassica oleracea line according to claim 7, characterized in that the NWB-CMS1 marker or NWB-CMS3 marker is amplified with a primer set consisting of a nucleotide sequence of SEQ ID NO: 3 and SEQ ID NO: 4 or a primer set consisting of a nucleotide sequence of SEQ ID NO: 5 and SEQ ID NO: 6, respectively.

9. A seed of the plant of NWB-CMS Brassica oleracea line according to claim 5.

10. A method for producing a hybrid seed of NWB-CMS Brassica oleracea line having cytoplasmic male sterility comprising breeding of a male fertile Brassica oleracea as a subject for introduction with the NWB-CMS cabbage plant of claim 1 as a breeding line.

11. A hybrid seed of NWB-CMS Brassica oleracea line produced by the method of claim 10.
